(19) United States
(12) Patent Application Publication
(10) Pub. No.: US 2010/0036324 A1
(43) Pub. Date: Feb. 11, 2010

(54) TUBE HOLDER

(75) Inventors: Rory Smith, Crawley (GB); Jeanette Berry, Leatherhead (GB)

Correspondence Address: NIXON & VANDERHYE, PC, 901 NORTH GLEBE ROAD, 11TH FLOOR, ARLINGTON, VA, 22203, US

(73) Assignee: CLINIMED (HOLDINGS) LIMITED, Loudwater (GB)

(21) Appl. No.: 12/187,016

(22) Filed: Aug. 6, 2008

(51) Int. Cl. A61M 5/32

(52) U.S. Cl. 604/180

(57) ABSTRACT

A tube holder (1) for removeably securing a tube (19) to the skin of a patient comprising a plastics film (3) bonded to an adhesive material on a bodyside surface of the film (3); and a separate first (7,9) and second means for applying pressure to a tube (19) located in the holder (1) during use, wherein the first (7,9) and second means are bonded to a tube-side surface of the plastics film (3) and pressure can be applied, in use, to at least two opposing sides of the tube (19).

The present invention relates to a tube holder, for holding a tube such as a catheter or a drip to the body of a patient.

Tubes, such as catheters or those which pass to a patient from a drip, need to be attached to the body of a patient in such a way as to allow the patient to move whilst the tube remains attached. It is common to attach the tubes using adhesive bandages or plasters. To adjust the position of the tube the adhesive bandage would have to be removed and replaced. This often leads to poor positioning of the tube because any adjustment causes unnecessary inconvenience and discomfort to the patient and wastes bandages.

U.S. Pat. No. 4,165,748 discloses a device which is adhesively fixed to patient but also has a Velcro™ “bridge” which removeably fastens around a tube, such as a catheter. Use of the removable fastener allows the tube's position to be adjusted without removing the device from the patient's skin. However, it does not offer the security of an adhesive fastening. It has been found that when the patient is unable to monitor the tube holder, for example when asleep, a patient can easily disturb the removable fastener and so the tube becomes detached from the patient's body.

The present invention sets out to provide a tube holder which alleviates the problems described above by providing a pad which can be either permanently or removeably fixed to a patient's body.

A tube holder for removeably securing a tube to the skin of a patient comprising a plastics film bonded to an adhesive material on a bodyside surface of the pad; and a separate first and second means for applying pressure to a tube located in the holder during use, wherein the first and second means are bonded to a tube-side surface of the plastics film and pressure can be applied, in use, to at least two opposing sides of the tube.

By “sides” it s meant areas on the circumference of the tube.

Applying pressure to at least two sides of the tube ensures the stability of the tube when it is held against a patient's skin, for example when the patient is moving around or when the patient is asleep and unable to monitor the tube.

Preferably, at least one of the means for applying pressure to the tube comprises at least one tab extending from the tube-side surface of the plastics film to form, in use, an arch over the tube.

By forming an arch it is meant that the at least one tab forms a bridge over the tube.

Preferably, at least one of the means for applying pressure to the tube comprises a layer of adhesive.

The layer of adhesive may comprise any one of an acrylic adhesive, a hot melt adhesive or a silicon adhesive.

More preferably, the layer of adhesive is pressure-sensitive.

Preferably, the layer of adhesive is covered by a removeable release liner.

More preferably, the release liner is a silicone coated paper.

Preferably, at least one of the means for applying pressure to the tube comprises a male/female connecting means.

Preferably, at least one of the means for applying pressure to the tube comprises a hook and loop fastener.

More preferably, the hook and loop fastener is Velcro™.

A male/female connecting means for applying pressure to the tube, or a hook and loop fastener, for example Velcro™, allows the tube holder to be adjusted during use.

The invention will now be described by way of example with reference to the accompanying diagrammatic drawings, in which:-

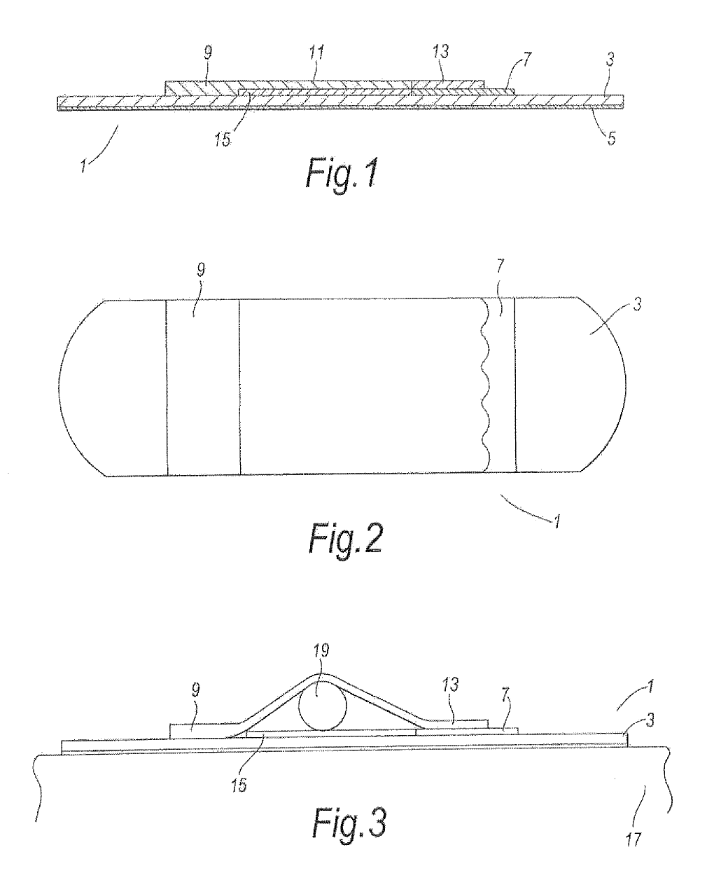

FIG. 1 is a cross sectional view of a tube holder constructed in accordance with the present invention;

FIG. 2 is a plan view from above of the tube holder of FIG. 1; and

FIG. 3 is a plan view from the side of the tube holder, in use, holding a tube.

As shown in FIG. 1, a tube holder 1 according to the invention comprises a thin adhesive pad 3. The dimensions of the pad 3 are chosen according to the tube with which it is to be used. For standard tubes the width of the pad may be 40 mm and length 140 mm or alternatively the width may be 25 mm and the length 110 mm. The pad 3 is formed of a thin plastics layer which is coated with a pressure sensitive adhesive on a body side surface of the pad 3. The pressure sensitive adhesive may be an acrylic adhesive, a silicon adhesive or a hot melt adhesive. The pressure sensitive adhesive layer is temporarily covered and protected by the use of a release liner 5 made of a plastics film, such as a silicone coated paper to be removed immediately prior to use of the tube holder 1. The release line 5 may be disposed of after it is removed or it may remain connected to the tube holder after it is removed from the adhesive layer to be reusable to recover the adhesive layer.

Two tabs 7, 9 are fixed by an adhesive to the opposing tube side surface of the thin adhesive pad 3. The first tab 7 is shorter than the second tab 9. The first tab 7 is adhesively fixed along the full length of one of its surfaces to tube side surface of the pad 3. The opposing surface of the first tab 7 comprises a fastening surface. The fastening surface is a hook and loop fastener such as Velcro™. The second tab 9 is a non-woven fabric such as polyurethane or an elastomeric material or a Lycra™-based polymer. Alternatively, the tab 9 may be made of a knitted fabric. The second tab 9 is fixed only along a minor portion of its length to the surface of the pad 3. The remaining length of the second tab comprises a section of hook and loop fastener 13 such as Velcro™.

Between the first and second tabs 7,9 there is central section of the tube side surface of the pad 3 which comprises an adhesive layer temporarily covered and protected by a release liner 15 made of a plastics film.

In use, as shown in FIG. 3, the release liner 5 on the body side surface of the adhesive pad 3 is removed and the pad 3 is adhesively fixed to a user's skin 17. If a tube is to be temporarily held by the tube holder 1, the tube 19 is placed over the release liner 15 of the central section. The second longer tab 9 is then looped over the tube such that the Velcro™ section 13 of the second tab 9 meets and is removeably fixed to the Velcro™ section of the first shorter tab 7 to “bridge” the tube 19 and so hold the tube in place. If a user wishes to re-position the tube 19, the Velcro™ section 13 of the second tab 9 is pulled from the first tab 7 and the process, which is described above, is repeated.

If the tube 19 is to be permanently held by the tube holder 1 the release liner 5 on the body side surface of the adhesive pad 3 is removed and the pad 3 is adhesively fixed to a user's skin 17. The release liner 15 is removed from the central section of the tube side surface of the pad 3. The tube 19 is placed over and so adhesively fixed to the pad 3. The second longer tab 9 is then looped over to “bridge” the tube such that the Velcro™ section of the second tab 13 meets and is removeably fixed to the Velcro™ section of the first shorter tab 7 to further secure the tube 17 in place. If a user wishes to reposition the tube 19 the tube holder 1 would have to be removed from the user's skin and replaced.

The above described embodiment has been given by way of example only, and the skilled reader will naturally appreciate that many variations could be made thereto without departing from the scope of the present invention.

1. A tube holder for removeably securing a tube to the skin of a patient comprising:
a plastics film having an adhesive material bonded on a bodyside surface of the film;
a first fastener bonded to a tube-side surface of the plastics film for applying pressure to a tube located in the holder during use, wherein the first fastener forms, in use, an arch on the tube side surface of the plastics film;
a second fastener comprising an adhesive layer on the tube-side surface of the plastic film, adjacent to a site where the first fastener is bonded to the tube-side surface of the plastics film; and
a first removable release liner overlying the adhesive layer on the tube-side surface of the plastic film, wherein the first release liner is selectively removable to expose the adhesive layer thereunder, whereby the tube can be temporarily held by the tube holder if the tube is placed on the first release liner and adhesively secured to the tube holder if the first release liner is removed before tube placement.

2. A tube holder according to claim 1, wherein the first fastener comprises at least one tab extending from the tube-side surface of the plastics film to form, in use, an arch over a tube.

3. (canceled)

4. A tube holder according to claim 1, wherein at least one of 1) the adhesive material on the bodyside surface of the plastics film and 2) the adhesive layer on the tube-side surface of the plastics film comprises an acrylic adhesive.

5. A tube holder according to claim 1, wherein at least one of 1) the adhesive material on the bodyside surface of the plastics film and 2) the adhesive layer on the tube-side surface of the plastics film comprises a hot melt adhesive.

6. A tube holder according to claim 1, wherein at least one of 1) the adhesive material on the bodyside surface of the plastics film and 2) the adhesive layer on the tube-side surface of the plastics film comprises silicon adhesive.

7. A tube holder according to claim 1, wherein at least one of 1) the adhesive material on the bodyside of the plastics film and 2) the adhesive layer on the tube-side surface of the plastics film is pressure-sensitive.

8. A tube holder according to claim 1, wherein the adhesive material on the bodyside surface of the plastics film is covered by a second removable release liner.

9. A tube holder according to claim 8, wherein the second release liner is a silicone coated paper.

10. A tube holder according to claim 1, wherein the first fastener comprises a male/female connecting means.

11. A tube holder according to claim 1 wherein the first fastener comprises a hook and loop fastener.

12. A tube holder according to claim 11 wherein the hook and loop fastener is Velcro™.

13. (canceled)

14. A tube holder according to claim 1, wherein the first fastener comprises first and second fastener components bonded to the tube-side surface of the plastics film, wherein the first fastener component forms, in use, an arch on the tube side surface of the plastics film and is selectively detachably secured to the second fastener component; and wherein the adhesive layer is disposed between sites where the first and second fastener components are bonded to the tube-side surface of the plastics film.
